SEQUENCE LISTING (12 sequences)

SEQ ID NO: 1 - DNA, 77 bp, synthetic construct
  source 1..77: mol_type = other DNA
gaacgacatggctacgagctcacagaacgacatggctacgatccgacttgctagaaggtcatggatgtgtgtcatcc

SEQ ID NO: 2 - DNA, 54 bp, synthetic construct
  source 1..54: mol_type = other DNA
aagtcggaggccaagcggtcttaggaagacaattctatcctgtcaggatgacac

SEQ ID NO: 3 - DNA, 40 bp, synthetic construct
  source 1..40: mol_type = other DNA
gctagaagnnnnnnnnnngcttggagtctctagtatcagt

SEQ ID NO: 4 - DNA, 17 bp, synthetic construct
  source 1..17: mol_type = other DNA
gaacgacatggctacga

SEQ ID NO: 5 - DNA, 22 bp, synthetic construct
  source 1..22: mol_type = other DNA
actgatactagagactccaagc

SEQ ID NO: 6 - DNA, 47 bp, synthetic construct
  source 1..47: mol_type = other DNA
tgtgagccaaggagttgatcgccagactgatactagagactccaagc

SEQ ID NO: 7 - DNA, 77 bp, synthetic construct
  source 1..77: mol_type = other DNA
gaacgacatggctacgagctcacagaacgacatggctacgatccgacttgcatgtaagtcatggatgtgtgtcatcc

SEQ ID NO: 8 - DNA, 54 bp, synthetic construct
  source 1..54: mol_type = other DNA
aagtcggaggccaagcggtcttaggaagacaaggattgactgtcaggatgacac

SEQ ID NO: 9 - DNA, 82 bp, synthetic construct
  source 1..82: mol_type = other DNA
tgacaggatagaattgtcttcctaagaccgcttggcctccgacttnnnnnnnnnnnnnnngcttggagtctctagtatcagt

SEQ ID NO: 10 - DNA, 82 bp, synthetic construct
  source 1..82: mol_type = other DNA
tgacagtcaatccttgtcttcctaagaccgcttggcctccgacttnnnnnnnnnnnnnnngcttggagtctctagtatcagt

SEQ ID NO: 11 - DNA, 21 bp, synthetic construct
  source 1..21: mol_type = other DNA
gctcacagaacgacatggcta

SEQ ID NO: 12 - DNA, 22 bp, synthetic construct
  source 1..22: mol_type = other DNA
actgatactagagactccaagc

(19) United States
(12) Patent Application Publication
(10) Pub. No.: US 2026/0159885 A1
(43) Pub. Date: Jun. 11, 2026

(54) HIGH-THROUGHPUT PROTEIN DETECTION METHOD BASED ON ADJACENT DNA ENCODING

(71) Applicant: MGI TECH CO., LTD., Shenzhen (CN)

(72) Inventors: Xigao CHEN, Shenzhen (CN); Huan MA, Shenzhen (CN); Renjie CHEN, Shenzhen (CN); Ming NI, Shenzhen (CN)

(21) Appl. No.: 19/537,575

(22) Filed: Feb. 12, 2026

Related U.S. Application Data
(63) Continuation of application No. PCT/CN2023/118631, filed on Sep. 13, 2023.

(51) Int. Cl. C12Q 1/6874; C12Q 1/6804; C12Q 1/6806; C12Q 1/6855; C12Q 1/686; G01N 33/68

(52) U.S. Cl. CPC C12Q 1/6874; C12Q 1/6804; C12Q 1/6806; C12Q 1/6855; C12Q 1/686; G01N 33/6803

(57) ABSTRACT

Provided is a probe composition, including: a first probe including a first oligonucleotide strand and a first antibody, 5′ end of the first oligonucleotide strand linked to the first antibody, and the first antibody having an activity of binding to a predetermined protein; a second probe including a second oligonucleotide strand and a second antibody, 5′ end of the second oligonucleotide strand linked to the second antibody, the second antibody having an activity of binding to the predetermined protein, and at least a partial sequence at 3′ end of the first oligonucleotide strand capable of reverse complementary pairing with at least a partial sequence at 3′ end of the second oligonucleotide strand; and a third probe including a third oligonucleotide strand, at least a partial sequence at 5′ end of the third oligonucleotide strand complementarily paired with a partial sequence at 3′ end of the second oligonucleotide strand.

### CROSS-REFERENCE TO RELATED APPLICATIONS

The present disclosure is a continuation of International Patent Application No. PCT/CN2023/118631 filed on Sep. 13, 2023, the entire contents of which are incorporated herein by reference.

### STATEMENT REGARDING SEQUENCE LISTING

A Sequence Listing associated with this application is being filed concurrently herewith in ASCII format and is hereby incorporated by reference into the present specification. The text file containing the Sequence listing is titled “Sequence_Listing.xml”, was created on Feb. 4, 2026, and is 11,591 bytes in size.

### FIELD

The present disclosure relates to the field of biotechnology. Specifically, the present disclosure relates to a high-throughput protein detection method based on proximity DNA encoding.

### BACKGROUND

Proteins are primary carriers of biological functions and can serve as chemical catalysts, structural components, and mediators of physiological processes. Research and techniques capable of accurately identifying and quantifying proteins can greatly advance the understanding of biology. Proteomics, as a core component of life science research in the post-genomic era, requires appropriate protein sequencing technologies in order to achieve an in-depth understanding of proteins and to further elucidate the molecular mechanisms underlying life activities and disease pathogenesis. However, unlike DNA or RNA, proteins possess complex structures and undergo extensive post-translational modifications. Moreover, the abundance of different proteins varies dramatically, spanning up to ten orders of magnitude. Many critical proteins are present at very low abundance, rendering their detection particularly challenging.

Existing proteomic technologies are generally classified into two major categories: mass spectrometry-based techniques and immuno-based (antibody-based) omics detection technologies. Mass spectrometry, as a fundamental detection technology, is suitable for large-scale exploratory research but requires relatively high abundance of a protein to be detected. In contrast, antibody-based targeted detection techniques are suitable for highly sensitive, highly selective, and quantitatively precise detection of predefined target proteins with good reproducibility, but such techniques are limited by antibody availability, resulting in restricted coverage and throughput. Other approaches, such as fluorescence labeling and mass spectrometry, can also sequence or quantify proteins within certain abundance ranges, but existing technologies still suffer from various limitations. Specifically, it remains difficult for current methods to simultaneously achieve ease of use, high throughput, broad coverage, and high sensitivity. The ability to simply, rapidly, and reproducibly measure target proteins spanning multiple orders of magnitude in abundance, as well as to routinely and comprehensively quantify proteomes in large sample cohorts, has yet to be fully realized.

Blood has long been widely used in clinical diagnosis of cancer and various others diseases. Blood contains more than ten thousand proteins, among which about 5,000 proteins have been identified, yet only about 150 proteins are currently used for disease diagnosis. Therefore, a more sensitive and higher-throughput method is required to screen biomarkers in blood.

Accordingly, there is a need to develop a new protein detection method with high throughput and high sensitivity, in order to enable faster and more effective disease detection and evaluation of therapeutic efficacy.

### SUMMARY

The present disclosure aims to solve, at least in part, one of the technical problems in the related art.

In a first aspect of the present disclosure, the present disclosure provides a probe composition. According to an embodiment of the present disclosure, the probe composition includes: a first probe including a first oligonucleotide strand and a first antibody, in which the 5′ end of the first oligonucleotide strand is linked to the first antibody, and the first antibody has an activity of binding to a predetermined protein; a second probe including a second oligonucleotide strand and a second antibody, in which the 5′ end of the second oligonucleotide strand is linked to the second antibody, the second antibody has an activity of binding to the predetermined protein, and at least a partial sequence at the 3′ end of the first oligonucleotide strand is capable of reverse complementary pairing with at least a partial sequence at the 3′ end of the second oligonucleotide strand; and a third probe including a third oligonucleotide strand, in which at least a partial sequence at the 5′ end of the third oligonucleotide strand is complementarily paired with a partial sequence at the 3′ end of the second oligonucleotide strand.

According to an embodiment of the present disclosure, the use of the probe composition in combination with a high-throughput sequencing method enables high-throughput and high-sensitivity detection of proteins.

It should be noted that, in the present disclosure, the terms “3′ end” and “5′ end” refer to the two end of an oligonucleotide strand. A “sequence at the 3′ end” refers to the nucleotide sequence starting from a nucleotide corresponding to the terminus of the 3′ end as a first nucleotide and extending consecutively therefrom, and may extend at most to a nucleotide corresponding to the 5′ end of the oligonucleotide strand. A “sequence at the 5′ end” of the oligonucleotide strand refers to a nucleotide sequence starting from a nucleotide corresponding to the terminus of the 5′ end as the first nucleotide and extending consecutively therefrom, and may extend at most to a nucleotide corresponding to the 3′ end of the oligonucleotide strand.

It should be noted that the second oligonucleotide strand includes at least a partial sequence complementary to the first oligonucleotide strand and a partial sequence complementary to the third oligonucleotide strand.

According to an embodiment of the present disclosure, the above probe composition may further include at least one of the following additional technical features:

According to an embodiment of the present disclosure, the 3′ end of the third oligonucleotide strand is linked to an affinity label.

According to an embodiment of the present disclosure, the affinity label is at least one selected from an antibody, biotin, a magnetic bead, and a gel bead.

According to a specific embodiment of the present disclosure, the 5′ end of the first oligonucleotide strand is linked to the first antibody.

According to a specific embodiment of the present disclosure, the 5′ end of the second oligonucleotide strand is linked to the second antibody.

According to a specific embodiment of the present disclosure, the 3′ end of the third oligonucleotide strand is linked to a third antibody.

According to an embodiment of the present disclosure, the first antibody has an activity of binding to a first site (i.e., epitope 1) of the predetermined protein, and the second antibody has an activity of binding to a second site (i.e., epitope 2) of the predetermined protein. The first site is different from the second site. Specifically, the first antibody and the second antibody bind to the same protein but at different binding sites.

According to an embodiment of the present disclosure, the first antibody has an activity of binding to the first site (i.e., epitope 1) of the predetermined protein, the second antibody has an activity of binding to the second site (epitope 2) of the predetermined protein, and the third antibody has an activity of binding to a third site (epitope 3) of the predetermined protein. The first site, the second site, and the third site are different from one another. Specifically, the first antibody, the second antibody, and the third antibody bind to the same protein but at different binding sites.

According to a specific embodiment of the present disclosure, the first antibody has an activity of binding to a first predetermined protein, and the second antibody has an activity of binding to a second predetermined protein. Specifically, the first protein is different from the second protein.

According to a specific embodiment of the present disclosure, the first antibody has an activity of binding to a first predetermined protein, the second antibody has an activity of binding to a second predetermined protein, and the third antibody has an activity of binding to a third predetermined protein. Specifically, the first protein, the second protein, and the third protein may be the same or different from one other.

According to a specific embodiment of the present disclosure, the third protein can be used for enrichment or purification of the probe composition.

According to an embodiment of the present disclosure, the sequence of the 3′ end of the first oligonucleotide strand is reverse complementary paired with at least a partial sequence at the 3′ end of the second oligonucleotide strand over a length ranging from 8 bp to 12 bp.

It should be noted that the 3′ end (a first complementary region) of the first oligonucleotide strand may be complementarily paired with a blocking sequence (cDNA1), for preventing binding between the first probe and the second probe.

According to an embodiment of the present disclosure, a length of complementary pairing between the 3′ end of the first oligonucleotide strand and the 3′ end of the second oligonucleotide strand may be 8 bp, 9 bp, 10 bp, 11 bp, or 12 bp. The inventors have found that the length of the complementary pairing sequence can be designed based on specific experimental requirements, and is not limited herein. Preferably, the complementary pairing sequence is relatively short, so as to reduce hybridization between the first oligonucleotide strand and the second oligonucleotide strand that occurs independently of antigen binding, thereby avoiding false-positive signals in protein detection.

According to an embodiment of the present disclosure, the first oligonucleotide strand includes a forward primer binding region, a random read region, a first protein barcode region, and a first complementary region.

According to a specific embodiment of the present disclosure, the forward primer binding region, the random read region, the first protein barcode region, and the first complementary region are not directly linked to one another. A spacer sequence is present between individual functional regions. A length of the spacer sequence can be designed as needed.

According to an embodiment of the present disclosure, the random read region includes a sequencing primer region.

According to an embodiment of the present disclosure, the random read region includes a sample index sequence.

According to an embodiment of the present disclosure, a forward primer may be the same as or different from a sequencing primer.

According to a specific embodiment of the present disclosure, the forward primer binding region is used for binding with a forward primer use in an amplification reaction. The sequencing primer region refers to a primer binding region during q-PCR or next-generation sequencing (NGS). The protein barcode region refers to a tag sequence used to distinguish different proteins. The first complementary region refers to a complementary region between the first oligonucleotide strand and the second oligonucleotide strand. The sample index sequence is used to distinguish different biological samples. In some embodiments, the arrangement order and the length of each functional region of the first oligonucleotide strand can be adjusted based on experimental requirements and are not particularly limited.

According to an embodiment of the present disclosure, the forward primer binding region includes an Ad153-F (BGISEQ/MGISEQ adapter) element.

According to an embodiment of the present disclosure, the sequencing primer region includes a sequencing primer binding region Rd1SP (Read 1 sequencing primer, a binding site for a first-round sequencing primer) element.

According to an embodiment of the present disclosure, the first protein barcode region includes an FBC (Forward Barcode) element.

According to an embodiment of the present disclosure, the first complementary region includes a Hyb (Hybridization) element.

According to an embodiment of the present disclosure, the second oligonucleotide strand includes a second complementary region. The second complementary region includes a 12-complementary region and a 23-complementary region.

According to a specific embodiment of the present disclosure, the 5′ end of the 12-complementary region is linked to the 3′ end of the 23-complementary region.

According to a specific embodiment of the present disclosure, the 12-complementary region is not directly linked to the 3′ end of the 23-complementary region. A spacer sequence is present between the two functional regions. A length of the spacer sequence can be designed based on experimental requirements.

According to an embodiment of the present disclosure, the 12-complementary region is reverse complementary to the first complementary region. According to a specific embodiment of the present disclosure, a schematic structural diagram of the second oligonucleotide strand is illustrated in FIG. 2.

According to an embodiment of the present disclosure, the 12-complementary region includes a Hyb element.

According to an embodiment of the present disclosure, the 23-complementary region includes a second barcode RBC (Reverse Barcode) element and an RS2 element.

According to an embodiment of the present disclosure, the third oligonucleotide strand at least includes a third complementary region, a molecular identifier (also known as unique molecular identifier, UMI), and a reverse primer binding region (Primer-1).

According to an embodiment of the present disclosure, the third oligonucleotide strand includes a sequencing primer binding region. The sequencing primer binding region may be the same as or different from the reverse primer binding region.

According to an embodiment of the present disclosure, the third oligonucleotide strand includes a second barcode region. The second barcode region may be used to distinguish different biological samples.

According to a specific embodiment of the present disclosure, the 3′ end of the third complementary region is linked to the 5′ end of the molecular identifier, and the 3′ end of the molecular identifier is linked to the 5′ end of the reverse primer binding region. A schematic structural diagram of the third oligonucleotide strand is illustrated in FIG. 3.

According to a specific embodiment of the present disclosure, the third complementary region, the molecular identifier, and the reverse primer binding region are not directly linked. A spacer sequence is present between individual functional regions. A length of the spacer sequence can be designed based on experimental requirements.

According to an embodiment of the present disclosure, the third complementary region includes at least one of an RBC element or an S2 element.

According to an embodiment of the present disclosure, the molecular identifier is a random sequence.

According to an embodiment of the present disclosure, the molecular identifier has a length ranging from 10 bp to 20 bp. The molecular identifier is a randomly synthesized oligonucleotide. According to an embodiment of the present disclosure, the inventors have found that, during protein detection in the related art, signal amplification through multiple rounds of PCR (e.g., 35 cycles) inevitably leads to signal bias caused by non-linear amplification. Therefore, the inventors introduce a molecular identifier (UMI) sequence to avoid signal bias arising from multiple rounds of PCR.

It should be noted that the UMI sequence is used to label molecules in a sample prior to sequencing. During a signal processing stage, duplicated signals generated during PCR can be normalized, which therefore avoids bias introduced by PCR amplification and sequencing redundancy and improving quantitative accuracy.

According to an embodiment of the present disclosure, the first complementary region is capable of complementary pairing with a 12-complementary region of the second complementary region, and the third complementary region is capable of complementary pairing with a 23-complementary region of the second complementary region.

According to an embodiment of the present disclosure, a Hyb element of the first complementary region is capable of complementary pairing with a Hyb element of the 12-complementary region.

According to an embodiment of the present disclosure, an RBC element of the third complementary region is capable of complementary pairing with an RRBC element of the 23-complementary region.

According to an embodiment of the present disclosure, an S2 element of the third complementary region is capable of complementary pairing with an RS2 element of the 23-complementary region.

According to a specific embodiment of the present disclosure, the first oligonucleotide strand, the second oligonucleotide strand, and the third oligonucleotide strand after complementary pairing are illustrated in FIG. 4.

According to another specific embodiment of the present disclosure, the first oligonucleotide strand, the second oligonucleotide strand, and the third oligonucleotide strand after complementary pairing are illustrated in FIG. 7 or FIG. 8 (lower panel).

In a second aspect of the present disclosure, the present disclosure provides a kit. According to an embodiment of the present disclosure, the kit includes the probe composition as described in the first aspect of the present disclosure and a ligase. According to an embodiment of the present disclosure, the kit is used for a protein detection and has advantages such as portability and low costs.

According to an embodiment of the present disclosure, the above kit further includes at least one of the following additional technical features.

According to an embodiment of the present disclosure, the kit further includes at least one selected from a primer, a DNA polymerase, a buffer solution, and dNTPs.

According to an embodiment of the present disclosure, the kit may further include at least one of an adapter, a probe, a sample index, and a protein barcode.

It should be noted that the sample index is a DNA sequence or a molecular marker used to label different samples. A sample index library refers to a collection of a series of sample indexes. A size of the library depends on detection throughput. A function of the sample index is to distinguish different samples, enabling parallel processing of samples during sequencing and ultimately assigning sequencing reads correctly to corresponding samples. The sample index is selected from at least one sample index in the sample index library.

It should be noted that the protein barcode, including FBC and RBC, refers to a DNA sequence or a molecular marker used to label different protein to be detected. A protein barcode library refers to a collection of a series of protein barcodes. A size of the library depends on the number of proteins to be detected. A function of the protein barcode is to distinguish different proteins to be detected, enabling parallel processing of proteins during sequencing and ultimately assigning sequencing reads correctly to corresponding proteins. The protein barcode is selected from at least one protein barcode in the protein barcode library.

According to an embodiment of the present disclosure, the adapter is Ad153-F (BGISEQ/MGISEQ adapter).

In a third aspect of the present disclosure, the present disclosure provides a method for detecting a protein. According to an embodiment of the present disclosure, the method includes: mixing a first probe, a second probe, and a third probe with a protein to be detected to obtain a sequencing library; and sequencing the sequencing library to detect the protein to be detected. The first probe includes a first oligonucleotide strand and a first antibody. The 5′ end of the first oligonucleotide strand is linked to the first antibody. The first antibody has an activity of binding to a predetermined protein. The second probe includes a second oligonucleotide strand and a second antibody. The 5′ end of the second oligonucleotide strand is linked to the second antibody. The second antibody has an activity of binding to the predetermined protein. At least a partial sequence at the 3′ end of the first oligonucleotide strand is capable of reverse complementary pairing with at least a partial sequence at the 3′ end of the second oligonucleotide strand. The third probe includes a third oligonucleotide strand. At least a partial sequence at the 5′ end of the third oligonucleotide strand is complementarily paired with a partial sequence at the 3′ end of the second oligonucleotide strand.

According to an embodiment of the present disclosure, protein detection using the above method can effectively reduce background signals and improve detection accuracy.

According to an embodiment of the present disclosure, the above method for detecting the protein further includes at least one of the following additional technical features.

According to an embodiment of the present disclosure, the method for detecting the protein further includes a ligation treatment for generating a nucleic acid sequence including a sequence of the first probe and a sequence of the third probe. In some embodiments of the present disclosure, only the ligation treatment is performed based on experimental requirements.

According to an embodiment of the present disclosure, the method for detecting the protein further includes an extension treatment and a ligation treatment, for generating a nucleic acid sequence including a sequence of the first probe and a sequence of the third probe. In some other embodiments of the present disclosure, the extension treatment may be added prior to the ligation treatment based on experimental requirements.

According to an embodiment of the present disclosure, the 3′ end of the third oligonucleotide strand is linked to an affinity label. The affinity label is at least one selected from an antibody, biotin, a magnetic bead, and a gel bead.

According to an embodiment of the present disclosure, the ligation treatment further includes at least one of: a: amplifying a product of the ligation treatment using a primer; or b: capturing the product of the ligation treatment using an affinity label.

According to an embodiment of the present disclosure, the mixing is performed by: performing a first mixing treatment on the first probe, the second probe, and the protein to be detected; and performing a hybridization treatment on a product of the first mixing treatment and the third probe.

According to a specific embodiment of the present disclosure, the first antibody of the first probe is capable of binding to the protein to be detected, and the second antibody of the second probe is also capable of binding to the protein to be detected, thereby bringing the first probe and the second probe into proximity. Thus, at least a partial sequence at the 3′ end of the first oligonucleotide strand is complementarily paired with the at least a partial sequence at the 3′ end of the second oligonucleotide strand, forming a product of the first mixing treatment. The product of the first mixing treatment further undergoes hybridization and ligation with the third probe to form a sequencing library.

According to a specific embodiment of the present disclosure, the mixing is performed by: mixing the first probe, the second probe, the third probe, and the protein to be detected. A partial sequence at the 5′ end of the third oligonucleotide strand of the third probe is complementarily paired with at least another partial sequence at the 3′ end of the second oligonucleotide strand of the second probe to form a double-stranded region.

According to a specific embodiment of the present disclosure, in a third mixing treatment, the first probe, the second probe, the third probe, cDNA1, and the protein to be detected are mixed. At least a partial sequence at the 5′ end of the third oligonucleotide strand of the third probe is complementarily paired with at least at least another partial sequence at the 3′ end of the second oligonucleotide strand of the second probe to form a double-stranded region. At least a portion of cDNA1 forms a double strand with the first complementary region of the first probe. In this mixing treatment, the first antibody is capable of binding to the protein to be detected, and the second antibody is also capable of binding to the protein to be detected, while the first complementary region of the first probe is blocked by cDNA1 and cannot bind to the 12-complementary region of the second probe. Magnetic beads are then used to bind to a biotin group at the 3′ end of the third probe, capturing a first probe-protein to be detected-double-stranded complex, and unbound probes are removed by washing. cDNA2 is then added to the mixture, and cDNA2 binds complementarily to cDNA1, displacing cDNA1 from the first probe. As a result, the first probe is brought into proximity with a partial double-stranded complex formed by the second probe and the third probe, while a single-stranded region remains at the 3′ end of the second oligonucleotide strand in the partial double-stranded complex, i.e., the corresponding 12-complementary region. At least a partial sequence at the 3′ end of the first oligonucleotide strand is then complementarily paired with at least a partial sequence at the 3′ end of the second oligonucleotide strand, such that the first complementary region of the first oligonucleotide strand is complementarily paired with the 12-complementary region of the second oligonucleotide strand, and a ligase is used to ligate the first oligonucleotide strand and the third oligonucleotide strand into a single oligonucleotide strand.

According to an embodiment of the present disclosure, the ligation treatment is performed in the presence of a ligase.

According to an embodiment of the present disclosure, the ligase is a Taq DNA ligase.

According to an embodiment of the present disclosure, the capturing is performed in the presence of a streptavidin magnetic bead. The inventors have found that, high background signals exist in protein detection in the related art, and by using streptavidin magnetic beads for washing, background signals can be reduced.

According to a specific embodiment of the present disclosure, during incubation of the first probe, the second probe, and the protein to be detected, cDNA1 is added to bind to the first complementary region of the first probe, thereby preventing binding between the first probe and the second probe. Thus, hybridization between the first probe and the second probe that occurs independently of the protein to be detected is reduced. Subsequently, magnetic bead purification is performed to elute probes that have not formed a first probe-antigen-second probe complex. Thereafter, cDNA2 is used to elute cDNA1 from the first complementary region, enabling ligation between the first probe and the second probe. As a result, “false-positive” signals are reduced.

Additional aspects and advantages of the present disclosure will be provided at least in part in the following description, or will become apparent at least in part from the following description, or can be learned from practicing of the present disclosure.

### BRIEF DESCRIPTION OF THE DRAWINGS

The above and/or additional aspects and advantages of the present disclosure will become more apparent and readily understood from the following description of embodiments taken in conjunction with the accompanying drawings.

FIG. 1 is a schematic diagram of a first oligonucleotide strand according to an embodiment of the present disclosure.

FIG. 2 is a schematic diagram of a second oligonucleotide strand according to an embodiment of the present disclosure.

FIG. 3 is a schematic diagram of a third oligonucleotide strand according to an embodiment of the present disclosure.

FIG. 4 is a schematic diagram illustrating ligation of a first oligonucleotide strand, a second oligonucleotide strand, and a third oligonucleotide strand according to an embodiment of the present disclosure.

FIG. 5 is a schematic flowchart of protein detection scheme 1 according to an embodiment of the present disclosure.

FIG. 6 is a schematic flowchart of protein detection scheme 2 according to an embodiment of the present disclosure.

FIG. 7 is a structural schematic diagram of protein detection scheme 1 according to an embodiment of the present disclosure.

FIG. 8 is a structural schematic diagram of protein detection scheme 2 according to an embodiment of the present disclosure.

FIG. 9 is a schematic diagram of a library structure for protein detection scheme 1 according to an embodiment of the present disclosure.

FIG. 10 is a schematic diagram of a library structure for protein detection scheme 2 according to an embodiment of the present disclosure.

FIG. 11 is a graph showing detection results of Vascular Endothelial Growth Factor (VEGF) in a low concentration range according to an embodiment of the present disclosure.

FIG. 12 is a graph showing detection results of VEGF and C-C Motif Chemokine Ligand 2 (CCL2) in a high concentration range according to an embodiment of the present disclosure.

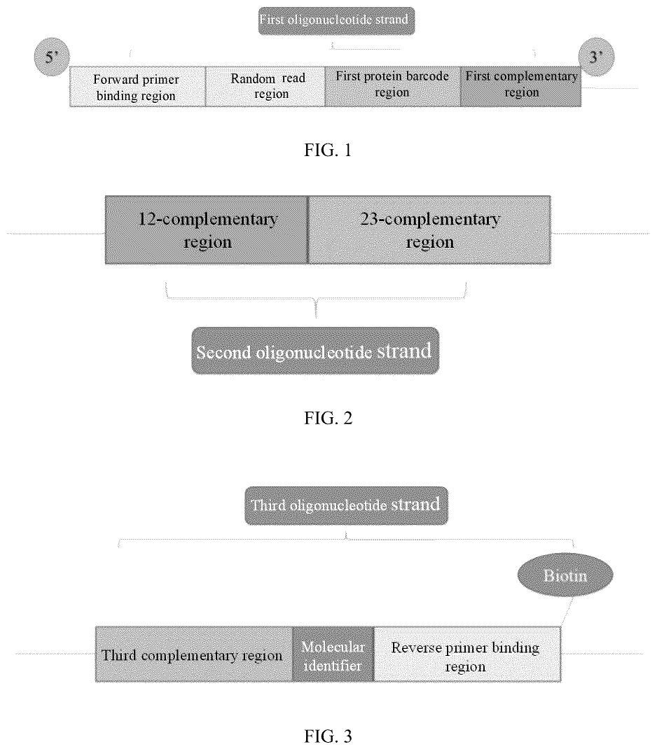

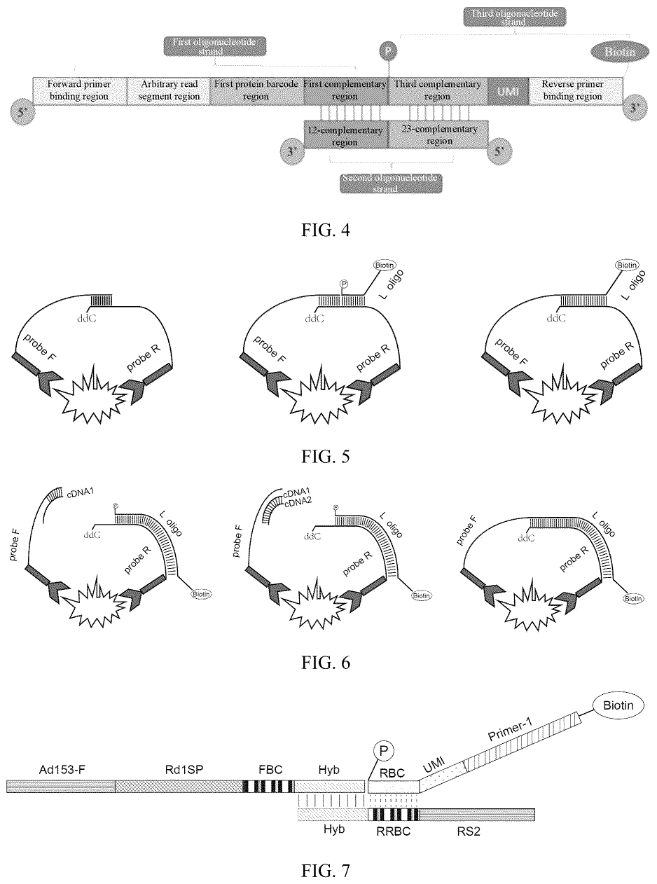

### DETAILED DESCRIPTION

Embodiments of the present disclosure will be described in detail below with reference to examples thereof as illustrated in the accompanying drawings, throughout which same or similar elements, or elements having same or similar functions, are denoted by same or similar reference numerals. The embodiments described below with reference to the drawings are illustrative only, and are intended to explain, rather than limit, the present disclosure.

### Definitions and Explanations

In the present disclosure, unless otherwise specified, singular forms such as “a,” “an,” etc., include plural referents (more than one); the term “a group” or “a plurality of” refers to two or more.

In the present disclosure, unless otherwise specified, terms “first,” “second,” “third,” “fourth,” etc., are only used for descriptive purposes, and cannot be understood as indicating or implying relative importance or implicitly indicating the number of indicated technical features. Therefore, the features associated with “first” and “second” may explicitly or implicitly include at least one of the features.

In the present disclosure, unless otherwise specified, the term “nucleotide” refers to four natural nucleotides (e.g., dATP, dCTP, dGTP, and dTTP, or ATP, CTP, GTP, and UTP) or their derivatives, and is sometimes directly represented by bases (A, T/U, C, G) it contains. Those skilled in the art can understand a specific meaning of a “nucleotide” or a “base” in a particular embodiment based on the context.

In the present disclosure, the term “sequencing” refers to sequence determination, synonymous with “nucleic acid sequencing” or “gene sequencing,” which denotes determination of the order of bases in a nucleic acid sequence. The term “sequencing” includes sequencing by synthesis (SBS) and/or sequencing by ligation (SBL); DNA sequencing and/or RNA sequencing; long-fragment sequencing and/or short-fragment sequencing, wherein “long” and “short” are relative terms, e.g., nucleic acid molecules longer than 1 Kb, 2 Kb, 5 Kb, or 10 Kb may be referred to as long fragments, while those shorter than 1 Kb or 800 bp may be referred to as short fragments; and paired-end sequencing, single-end sequencing, and/or mate-pair sequencing, wherein the terms “paired-end sequencing” or “mate-pair sequencing” refers to reading of any two non-overlapping segments or parts of the same nucleic acid molecule.

In the present disclosure, unless otherwise specified, the term “sequencing read” is used interchangeably with terms “read” and “read segment” and refers to a nucleic acid sequence obtained during sequencing. The nucleic acid sequence is referred to herein as a “sequencing read” or “read.”

In the present disclosure, sequencing platforms that may be used for the above sequencing method include, are but not limited to, Illumina's HiSeq/MiSeq/NextSeq/NovaSeq sequencing platforms; Thermo Fisher/Life Technologies' Ion Torrent platform; BGI's BGISEQ and MGISEQ/DNBSEQ platforms, and single-molecule sequencing platforms.

It should be noted that the term “protein” in the present disclosure includes antigens, polypeptides, etc.

It should be noted that sequences of elements described in the present disclosure are presented from the 5′ end to the 3′ end.

It should be noted that the term “linking” or “linked” in the present disclosure includes both direct linking or indirect linking.

### RELATED ART

Proximity Extension Assay (PEA) technology employs paired antibodies to recognize and capture a protein to be detected. Each antibody is conjugated with a nucleic acid sequence carrying encoding information corresponding to the protein, forming a pair of probes. Through orthogonal hybridization, extension, amplification, and sequencing of the nucleic acid sequence, signal amplification and readout are achieved. When combined with an NGS sequencing platform, this technology enables highly sensitive simultaneous detection with relatively large sample numbers (e.g., 96 samples) and high throughput (e.g., 384 marker combinations).

However, the existing technology still has the following deficiencies in protein detection.

On the one hand, the entire PEA detection process is carried out in a liquid phase and lacks an elution step to remove uncaptured antibodies, and oligo fragments carried by a forward encoding probe (probe F, conjugated with a forward oligo) and a reverse encoding probe (probe R, conjugated with a reverse oligo) that are conjugated with antibodies to carry protein encoding information contain complementary sequences. As a result, probe F and probe R undergo extensive hybridization independent of protein capture in the liquid phase, thereby generating “false-positive” signals.

On the other hand, due to differences in initial template concentrations, multiple rounds of PCR amplification lead to non-linear amplification, resulting in bias. Relying solely on a quality control system (e.g., amplification control or other amplification metrics) is insufficient for an effective correction.

### Protein Detection Method

In an aspect of the present disclosure, the present disclosure provides a protein detection method for reducing background signal intensity and improving sensitivity of protein detection. The protein detection method described in the present disclosure utilizes streptavidin magnetic beads to wash an antibody-protein-antibody complex, thereby removing complexes formed independently of proteins (including antigens). In another aspect, according to an embodiment of the present disclosure, a UMI sequence is introduced into a third oligonucleotide strand to facilitate correction and deduplication during subsequent library preparation and sequencing, thereby improving the accuracy of protein detection.

According to an embodiment of the present disclosure, the following are provided.

A first probe is provided. The first probe includes a first oligonucleotide strand and a first antibody. The 5′ end of the first oligonucleotide strand is linked to the first antibody. The first antibody has an activity of binding to a predetermined protein.

According to an embodiment of the present disclosure, the first oligonucleotide strand includes a forward primer binding region, a sequencing primer region, a first protein barcode region, and a first complementary region.

According to an embodiment of the present disclosure, the forward primer binding region includes an ad153-F element.

According to an embodiment of the present disclosure, the sequencing primer region includes an Rd1SP element.

According to an embodiment of the present disclosure, the first protein barcode region includes an FBC element.

According to an embodiment of the present disclosure, the first complementary region includes a Hyb element.

According to a specific embodiment of the present disclosure, in the first probe, the connection from left to right is as follows: the first antibody is linked to the 5′ end of the Ad153-F element, the 3′ end of the Ad153-F element is linked to the 5′ end of the Rd1SP element, the 3′ end of the Rd1SP element is linked to the 5′ end of the FBC element, and the 3′ end of the FBC element is linked to the 5′ end of the Hyb element.

A second probe is provided. The second probe includes a second oligonucleotide strand and a second antibody. The 5′ end of the second oligonucleotide strand is linked to the second antibody. The second antibody has an activity of binding to the predetermined protein. At least a partial sequence at the 3′ end of the first oligonucleotide strand is capable of reverse complementary pairing with at least a partial sequence at the 3′ end of the second oligonucleotide strand.

According to an embodiment of the present disclosure, the second oligonucleotide strand includes a second complementary region. The second complementary region includes a 12-complementary region and a 23-complementary region.

According to an embodiment of the present disclosure, the 12-complementary region includes a Hyb element.

According to an embodiment of the present disclosure, the 23-complementary region includes at least one of an RRBC element or an RS2 element.

According to a specific embodiment of the present disclosure, in the second probe, the connection from left to right is as follows: the 3′ end of the Hyb element is modified with ddC, the 5′ end of the Hyb element is linked to the 3′ end of the RRBC element, the 5′ end of the RRBC element is linked to the 3′ end of the RS2 element, and the 5′ end of the RS2 element is linked to the second antibody.

A third probe is provided. The third probe includes a third oligonucleotide strand. At least a partial sequence at the 5′ end of the third oligonucleotide strand is complementarily paired with a partial sequence at the 3′ end of the second oligonucleotide strand.

According to a specific embodiment of the present disclosure, the 3′ end of the third oligonucleotide strand is linked to biotin.

According to an embodiment of the present disclosure, the third oligonucleotide strand includes a third complementary region, a molecular identifier (UMI), and a reverse primer binding region.

According to an embodiment of the present disclosure, the third complementary region includes at least one of an RBC element or an S2 element.

According to an embodiment of the present disclosure, the molecular identifier (UMI) is a randomly synthesized oligonucleotide sequence having a length ranging from 10 bp to 20 bp.

According to an embodiment of the present disclosure, the reverse primer binding region includes a primer-1 element.

According to a specific embodiment of the present disclosure, in the third probe, the connection from left to right is as follows: the 3′ end of the RBC element is linked to the 5′ end of the UMI element, the 3′ end of the UMI element is linked to the 5′ end of the primer-1 element, and the 3′ end of the primer-1 element is linked to the biotin; or the 3′ end of the RBC element is linked to the 5′ end of the S2 element, the 3′ end of the S2 element is linked to the 5′ end of the UMI element, the 3′ end of the UMI element is linked to the 5′ end of the primer-1 element, and the 3′ end of the primer-1 element is linked to the biotin.

According to an embodiment of the present disclosure, the method includes: capturing a protein to be detected using a probe composition, each of the first antibody and the second antibody having an activity of binding to the protein to be detected; constructing a sequencing library based on a product of the capturing; and performing sequencing on the sequencing library to detect the protein to be detected.

According to an embodiment of the present disclosure, a portion of cDNA1 (an oligonucleotide sequence complementary to a partial sequence of Probe F) is complementary to the 3′ end of the first probe to block binding between the first probe and the second probe. Subsequently, magnetic beads are used to capture a first probe-antigen-second probe complex, and the first probe not bound to the antigen is washed away. Then, cDNA2 (an oligonucleotide sequence complementary to a partial sequence of cDNA1) is added to competitively bind to cDNA1, eluting cDNA1 from the first probe, while the first probe binds to the second probe through complementary region at the 3′ end of the first probe.

Technical solutions of the present disclosure are described in detail below with reference to the accompanying drawings.

Protein detection scheme 1, with reference to FIG. 5, FIG. 7, and FIG. 9.

Incubation—probe capture of antigen (left panel of FIG. 5): Probe F (first probe) and probe R (second probe), each carrying oligo (an oligonucleotide strand), are incubated with an antigen. The specific antibody carried on the probes captures the antigen, and the probes undergo hybridization via complementary regions of the oligonucleotides.

Hybridization—hybridization of with probe R (middle panel of FIG. 5), L oligo (third probe) is then added to the incubation system. The terminus of the 3′ end of L oligo is modified with biotin and carried a UMI barcode sequence having a length ranging from 10 bp to 20 bp. L oligo binds to a partial region of the oligo of probe R.

Ligation—ligation of L oligo to probe F (right panel of FIG. 5): A ligation system then ligated L oligo to the oligo of probe F.

Further, the ligation system is purified using streptavidin magnetic beads to remove probes that have not undergone ligation. The purified product is subjected to a PCR amplification to prepare a sequencing library (FIG. 11), followed by sequencing. The protein concentration in the sample is reflected based on signal intensity.

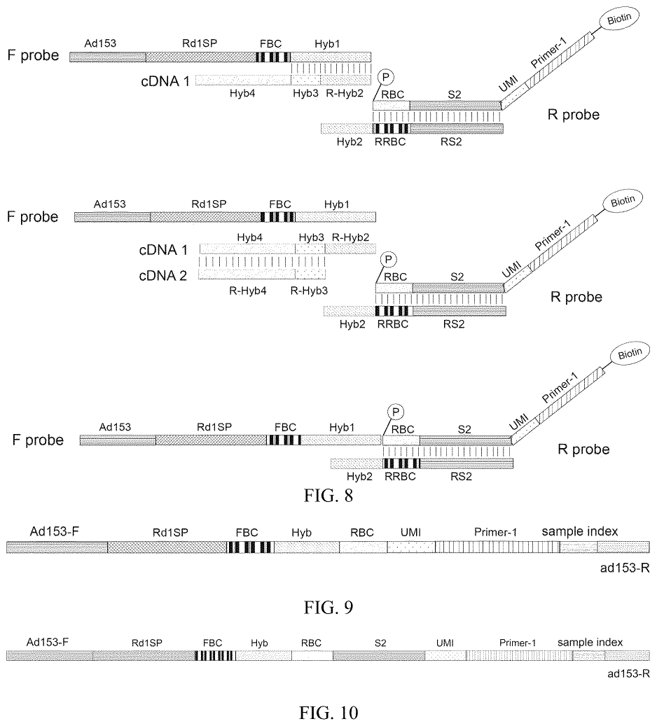

Protein detection scheme 2, with reference to FIG. 6, FIG. 8, and FIG. 10. According to an embodiment of the present disclosure, the method includes the following steps:

During preparation of probe F (first probe) and probe R (second probe), L oligo (third probe) is first hybridized with the oligo of probe R. The resulting double-stranded structure is then conjugated to an antibody.

Co-incubation of antibody, antigen, and cDNA1 (left panel of FIG. 6): probe F, probe R, cDNA1, and the antigen are incubated together. The specific antibody carried on the probes captures the antigen (upper panel of FIG. 8).

Removal of free probe F and displacement of cDNA1 by cDNA2 (middle panel of FIG. 6): A partial region of cDNA1 binds to a first complementary region of probe F. The probe F-antigen-probe R complex is purified using magnetic beads to remove free probe F. Subsequently, cDNA2 competitively hybridizes with cDNA1, thereby displacing and washing cDNA1 from probe F (middle panel of FIG. 8).

Ligation of L oligo to probe F (right panel of FIG. 6): Partial regions of probe F and probe R hybridize, and a ligase is added to the retained system to ligate the L oligo to the oligo of probe F (lower panel of FIG. 8).

Further, the ligation reaction system is further washed to remove impurities, and the purified product is subjected to q-PCR detection. The protein concentration in the sample is reflected based on signal intensity or expression level.

The present disclosure is illustrated below by way of examples, but it should not be understood that the scope of the subject matter of the present disclosure is limited to the following examples. Any technology implemented based on the above contents of the present disclosure falls within the scope of the present disclosure. Compounds or reagents used in the following examples are commercially available or can be prepared by conventional methods known to those skilled in the art. Experimental instruments used are commercially available.

### Example 1

This example corresponds to technical solution 1 of the present disclosure.

1. 20 μg of antibody (1 mg/mL) was reacted with DBCO-PEG5-NHS at a molar ratio of 1:10 at room temperature for 3 h. Excess DBCO-PEG5-NHS was removed using a Zeba Spin (desalting column). Then, F oligo was added at a ratio of antibody:oligo (oligonucleotide)=1:3. Partial oligo sequences are shown in Table 1.

TABLE 1
Oligo sequences

| Sequence ID | Sequence |
|---|---|
| F oligo for VEGF165 | /5azide/GAACGACATGGCTACGAGCTCACAGAAC |
| (forward oligonucleotide | GACATGGCTACGATCCGACTTGCTAGAAGGTCA |
| for VEGF165) | TGGATGTGTGTCATCC/3/ (SEQ ID NO: 1) |
| R oligo for VEGF165 | /5azide/AAGTCGGAGGCCAAGCGGTCTTAGGAAG |
| (reverse oligonucleotide | ACAATTCTATCCTGTCAGGATGACAC/3ddC/ |
| for VEGF165) | (SEQ ID NO: 2) |
| L oligo for VEGF165 | GCTAGAAGNNNNNNNNNNGCTTGGAGTCTCTA |
| (template oligonucleotide | GTATCAGT (SEQ ID NO: 3) |
| for VEGF165) | |
| Ad153 PCR2 1 (forward | /5Phos/GAACGACATGGCTACGA/3/ |
| primer) | (SEQ ID NO: 4) |
| Primer-1 (reverse primer) | ACTGATACTAGAGACTCCAAGC (SEQ ID NO: 5) |
| index Primer for sample 1 | TGTGAGCCAAGGAGTTGATCGCCAGACTGATAC |
| | TAGAGACTCCAAGC (SEQ ID NO: 6) |

2. 20 μg of antibody (1 mg/mL) was reacted with DBCO-PEG5-NHS at a molar ratio of 1:10 at room temperature for 3 h. Excess DBCO-PEG5-NHS was removed using a Zeba Spin. Then, R oligo was added at a ratio of antibody:oligo=1:3.

3. Six proteins were diluted at different ratios using a sample dilution buffer (0.1% BSA in PBS, pH 7.2) to prepare concentration gradients of 0 μg/mL to 750 μg/mL or 50 μg/mL to 1,000 μg/mL, thereby obtaining mixed protein samples (sample mix; sample 1 to sample 5).

4. 1 μL of diluted protein was mixed with 1 μL of blocking buffer (0.3 mg/ml blocking reagents, 100 μg/mL ssDNA, 0.1% BSA, 4 mM EDTA, 0.2% Triton X-100, 0.02% sodium azide), pipetted 10 times to mix thoroughly, and incubated at 25° C. for 20 min.

5. Forward probes (probe F) and reverse probes (probe R) corresponding to the six proteins were diluted to 100 μM using a probe dilution buffer (25 mM Tris-HCl pH 7.2, 5 mM EDTA, 1 mM biotin, 26 μg ssDNA, 0.01% sodium azide). 2 μL of the diluted probe mixture (probe mix) was added to the corresponding samples.

6. The mixture was pipetted 20 times to mix thoroughly and incubated at 4° C. for at least 16 h.

7. 46 μL of L ligation mixture (ligation mix, 1× HiFi Taq DNA ligase buffer), 1 μL HiFi Taq DNA ligase (NEB, M0647S), and L oligo corresponding to each probe (435 nM, XXXXXXXXNNNNNNNNNNGCTTGGAGTCTCTAGTATCAGT (SEQ ID NO: 3, 9 or 10), X: barcode; N: random sequence) was added to the incubation system, followed by incubation at 45° C. for 15 min.

8. 5 μL of streptavidin magnetic beads were washed three times with 1×BW (5 mM Tris-HCl, 0.5 mM EDTA, 1 M NaCl), resuspended in 250 μL of 2×BW, and evenly divided into five portions of 50 μL each.

9. The ligated samples were added to the streptavidin magnetic beads (Thermo, 11206D), incubated at the room temperature for 15 min, washed twice with 1×BW, and resuspended in 50 μL of TE buffer.

10. A first round of PCR was performed on the magnetic beads using a 20 μL PCR system including: 1× Thermo pol reaction buffer (NEB B9004S), 0.25 μM primers (forward primer: Ad153_PCR2_1 5′P-GAACGACATGGCTACGA-3′ (SEQ ID NO: 4); reverse primer: primer1 5′-ACTGATACTAGAGACTCCAAGC-3′ (SEQ ID NO: 5)), 250 μM dNTPs, 0.2 μL HotStart Taq (NEB M0495L), and 2 μL of magnetic bead sample. An amplification program was performed as follows: pre-denaturation at 98° C. for 3 min; 25 cycles of denaturation at 95° C. for 30 s, annealing at 60° C. for 30 s, and extension at 72° C. for 15 s; and an extension at 72° C. for 5 min.

11. A second round of PCR amplification was performed using a 20 μL PCR system including: 1× Thermo pol reaction buffer (NEB B9004S), 0.25 μM primers (forward primer: Ad153_PCR2_1 5′P-GAACGACATGGCTACGA-3′ (SEQ ID NO: 4); reverse primer: Index primer 5′-TGTGAGCCAAGGAGTTGATCGCCAGACTGATACTAGAGACTCCAAGC-3′ (SEQ ID NO: 6)), 250 μM dNTPs, 0.2 μL HotStart Taq (NEB M0495L), and 2 μL of the first-round PCR product. An amplification program was performed as follows: pre-denaturation at 98° C. for 3 min; 10 cycles of denaturation at 95° C. for 30 s, annealing at 60° C. for 30 s, and extension at 72° C. for 15 s; and an extension at 72° C. for 5 min.

12. DNBs were prepared using a one-step DNB preparation kit (940-000034-00). Specifically, 30 μL of DNB preparation reaction solution 1 was added to 10 μL of DNA sample. The mixture was denatured at 95° C. for 3 min, annealed at 57° C. for 3 min, and held at 4° C. Then, 22 μL of DNB preparation reaction solution 2 (DNB polymerase mix I and DNB polymerase mix II) was added to the reaction system, followed by reaction at 30° C. for 25 min and holding at 4° C. After DNB concentration was determined, sequencing was performed.

The detection results are shown in FIG. 11, illustrating detection of VEGF165 under low-concentration conditions (left) and high-concentration conditions (right). The results show that, under both concentration conditions, the measured signal intensity exhibits a positive linear correlation with antigen concentration. The detection range for VEGF at least ranges from 20 μg/mL to 800 μg/mL.

### Example 2

This example corresponds to technical solution 2 of the present disclosure.

1. 20 μg of antibody (1 mg/mL) was reacted with DBCO-PEG5-NHS at a molar ratio of 1:10 at room temperature for 3 h. Excess DBCO-PEG5-NHS was removed using a Zeba Spin. Then, F oligo was added at a ratio of antibody:oligo=1:3. Partial oligo sequences are shown in Table 2.

TABLE 2
Oligo sequences

| Sequence ID | Sequence |
|---|---|
| F oligo for VEGF165 | /5azide/GAACGACATGGCTACGAGCTCACAGAAC |
| (forward oligonucleotide | GACATGGCTACGATCCGACTTGCTAGAAGGTCA |
| for VEGF165) | TGGATGTGTGTCATCC/3/ (SEQ ID NO: 1) |
| R oligo for VEGF165 | /5azide/AAGTCGGAGGCCAAGCGGTCTTAGGAAG |
| (reverse oligonucleotide | ACAATTCTATCCTGTCAGGATGACAC /3ddC/ |
| for VEGF165) | (SEQ ID NO: 2) |
| F oligo for CCL2 (forward | /5azide/GAACGACATGGCTACGAGCTCACAGAAC |
| oligonucleotide for CCL2) | GACATGGCTACGATCCGACTTGCATGTAAGTCA |
| | TGGATGTGTGTCATCC/3/ (SEQ ID NO: 7) |
| R oligo for CCL2 (reverse | /5azide/AAGTCGGAGGCCAAGCGGTCTTAGGAAG |
| oligonucleotide for CCL2) | ACAAGGATTGACTGTCAGGATGACAC /3ddC/ |
| | (SEQ ID NO: 8) |
| L oligo for VEGF | /5Phos/TGACAGGATAGAATTGTCTTCCTAAGACC |
| | GCTTGGCCTCCGACTTNNNNNNNNNNNNNNNG |
| | CTTGGAGTCTCTAGTATCAGT/3biotin/ |
| | (SEQ ID NO: 9) |
| L oligo for CCL2 | /5Phos/TGACAGTCAATCCTTGTCTTCCTAAGACC |
| | GCTTGGCCTCCGACTTNNNNNNNNNNNNNNNG |
| | CTTGGAGTCTCTAGTATCAGT/3biotin/ |
| | (SEQ ID NO: 10) |
| q-PCR-F | GCTCACAGAACGACATGGCTA (SEQ ID NO: 11) |
| q-PCR-R | ACTGATACTAGAGACTCCAAGC (SEQ ID NO: 12) |

2. 20 μg (1 mg/mL) of antibody was reacted with DBCO-PEG5-NHS at a molar ratio of 1:10 at room temperature for 3 h. Excess DBCO-PEG5-NHS was removed using a Zeba Spin. L oligo and the oligo of probe R were mixed in TE at an equal molar ratio, incubated at 95° C. for 3 min, and slowly cooled to 25° C. at a rate of 0.5° C./s. Then, annealed double-stranded oligo was added at a ratio of antibody:oligo=1:3.

3. Protein samples were diluted using a sample dilution buffer (0.1% BSA in PBS, pH 7.2) to different concentrations of 0 μg/mL to 500 μg/mL.

4. 1 μL of the diluted sample was added to 1 μL of blocking buffer (0.3 mg/mL blocking reagents, 100 μg/mL ssDNA, 0.1% BSA, 4 mM EDTA, 0.2% TritonX-100, 0.02% sodium azide) and incubated at 25° C. for 20 min. Then, 2 μL of probe mix (25 mM Tris-HCl pH7.2, 5 mM EDTA, 1 mM biotin, 26 μg ssDNA, 0.01% sodium azide, 100 μM probe F, 100 pM probe R, 1 nM cDNA1) was added, followed by incubation at 4° C. for at least 16 h.

5. 5 μL of streptavidin magnetic beads were washed three times with 1×BW (5 mM Tris-HCl, 0.5 mM EDTA, 1 M NaCl), resuspended in 250 μL of 2×BW, and evenly divided into five portions of 50 μL each.

6. The incubated samples were added to the streptavidin magnetic beads (Thermo, 11206D), incubated at the room temperature for 15 min, and washed three times with 1×BW.

7. 20 μL of cDNA2 solution (1×TE, 10 nM cDNA2) was added to the incubation system, followed by incubation at 4° C. for 30 min.

8. The magnetic beads were washed once with 1×BW. Then, 20 μL of cDNA2 solution (1×TE, 10 nM cDNA2) was added again, followed by incubation at 4° C. for 30 min.

9. The magnetic beads were washed three times with 1×BW.

10. Ligation mix (1× HiFi Taq DNA ligase buffer and 1 μL HiFi Taq DNA ligase (NEB, M0647S)) was added, followed by incubation at 45° C. for 15 min.

11. The magnetic beads were washed once with 1×BW and resuspended in 20 μL of TE buffer.

12. The ligated samples were subjected to a two-step q-PCR detection (pre-denaturation at 95° C. for 30s; 40 cycles of denaturation at 95° C. for 5s, annealing at 60° C., and extension for 30s) using a 20 μL of q-PCR system including: 1 ×TB Green Premix Ex Taq II (TAKARA RR82WR), 0.4 μM primer (forward primer: q-PCR-F GCTCACAGAACGACATGGCTA (SEQ ID NO: 11), reverse primer: q-PCR-R ACTGATACTAGAGACTCCAAGC (SEQ ID NO: 12)), and 2 μL of the ligated samples.

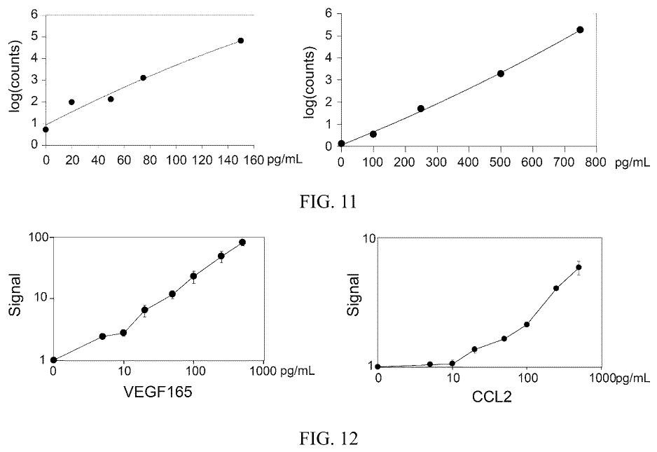

13. The detection results for VEGF and CCL2 are shown in FIG. 12. The results show that signal intensity is positively correlated with antigen concentration. The detection range can at least range from 5 μg/mL to 500 μg/mL for VEGF and from 10 μg/mL to 500 μg/mL for CCL2.

In addition, terms “first” and “second” are only used for descriptive purposes, and cannot be understood as indicating or implying relative importance or implicitly indicating the number of indicated technical features. Therefore, the features associated with “first” and “second” may explicitly or implicitly include at least one of the features. In the description of the present disclosure, “plurality” means at least two, unless otherwise specifically defined.

Reference throughout this specification to “an embodiment”, “some embodiments”, “an example”, “a specific example”, or “some examples” means that a particular feature, structure, material, or characteristic described in connection with the embodiment or example is included in at least one embodiment or example of the present disclosure. The appearances of the above phrases in various places throughout this specification are not necessarily referring to the same embodiment or example. Further, the particular features, structures, materials, or characteristics may be combined in any suitable manner in one or more embodiments or examples. In addition, different embodiments or examples and features of different embodiments or examples described in the specification may be combined by those skilled in the art without mutual contradiction.

Although embodiments of the present disclosure have been shown and described above, it should be understood that the above embodiments are merely exemplary, and cannot be construed to limit the present disclosure. For those skilled in the art, changes, alternatives, and modifications can be made to the embodiments without departing from the scope of the present disclosure.

What is claimed is:

1. A probe composition, comprising:
a first probe, comprising a first oligonucleotide strand and a first antibody, wherein the 5′ end of the first oligonucleotide strand is linked to the first antibody, and wherein the first antibody has an activity of binding to a predetermined protein;
a second probe, comprising a second oligonucleotide strand and a second antibody, wherein the 5′ end of the second oligonucleotide strand is linked to the second antibody, wherein the second antibody has an activity of binding to the predetermined protein, and wherein at least a partial sequence at the 3′ end of the first oligonucleotide strand is capable of reverse complementary pairing with at least a partial sequence at the 3′ end of the second oligonucleotide strand; and
a third probe, comprising a third oligonucleotide strand, wherein at least a partial sequence at the 5′ end of the third oligonucleotide strand is complementarily paired with a partial sequence at the 3′ end of the second oligonucleotide strand.

2. The probe composition according to claim 1, wherein the 3′ end of the third oligonucleotide strand is linked to an affinity label.

3. The probe composition according to claim 2, wherein the affinity label is at least one selected from an antibody, biotin, a magnetic bead, and a gel bead.

4. The probe composition according to claim 1, wherein the first antibody has an activity of binding to a first site of the predetermined protein, and wherein the second antibody has an activity of binding to a second site of the predetermined protein, the first site being different from the second site.

5. The probe composition according to claim 1, wherein the 3′ end of the first oligonucleotide strand is reverse complementary paired with at least a partial sequence at the 3′ end of the second oligonucleotide strand over a length ranging from 8 bp to 12 bp.

6. The probe composition according to claim 1, wherein the first oligonucleotide strand comprises a forward primer binding region, a random read region, a first protein barcode region, and a first complementary region.

7. The probe composition according to claim 1, wherein the second oligonucleotide strand comprises a second complementary region, the second complementary region comprising a 12-complementary region and a 23-complementary region,
optionally, the 12-complementary region is reverse complementary to the first complementary region.

8. The probe composition according to claim 1, wherein the third oligonucleotide strand comprises a third complementary region, a molecular identifier, and a reverse primer binding region.

9. The probe composition according to claim 7, wherein the first complementary region is capable of complementary pairing with the 12-complementary region of the second complementary region, and wherein the third complementary region is capable of complementary pairing with the 23-complementary region of the second complementary region.

10. A kit, comprising:
the probe composition according to claim 1; and
a ligase.

11. The kit according to claim 10, further comprising at least one selected from a primer, a DNA polymerase, a buffer solution, and dNTPs.

12. A method for detecting a protein, the method comprising:
mixing a first probe, a second probe, and a third probe with a protein to be detected, to obtain a sequencing library; and
sequencing the sequencing library for detecting the protein to be detected, wherein:
the first probe comprises a first oligonucleotide strand and a first antibody, wherein the 5′ end of the first oligonucleotide strand is linked to the first antibody, and wherein the first antibody has an activity of binding to a predetermined protein;
the second probe comprises a second oligonucleotide strand and a second antibody, wherein the 5′ end of the second oligonucleotide strand is linked to the second antibody, wherein the second antibody has an activity of binding to the predetermined protein, and wherein at least a partial sequence at the 3′ end of the first oligonucleotide strand is capable of reverse complementary pairing with at least a partial sequence at the 3′ end of the second oligonucleotide strand; and
the third probe comprises a third oligonucleotide strand, wherein at least a partial sequence at the 5′ end of the third oligonucleotide strand is complementarily paired with a partial sequence at the 3′ end of the second oligonucleotide strand.

13. The method according to claim 12, further comprising a ligation treatment for generating a nucleic acid sequence, the nucleic acid sequence comprising a sequence of the first probe and a sequence of the third probe.

14. The method according to claim 12, further comprising an extension treatment and a ligation treatment for generating a nucleic acid sequence, the nucleic acid sequence comprising a sequence of the first probe and a sequence of the third probe.

15. The method according to claim 12, wherein the 3′ end of the third oligonucleotide strand is linked to an affinity label, the affinity label being at least one selected from an antibody, biotin, a magnetic bead, and a gel bead.

16. The method according to claim 13, wherein the ligation treatment further comprises at least one of:
step a: amplifying a product of the ligation treatment using a primer; or
step b: capturing the product of the ligation treatment using an affinity label.

17. The method according to claim 12, wherein said mixing is performed by:
performing a first mixing treatment by mixing the first probe, the second probe, and the protein to be detected; and
performing a second mixing treatment by mixing the third probe with mixture obtained by the first mixing treatment.

18. The method according to claim 12, wherein said mixing is performed by mixing the first probe, the second probe, the third probe, and the protein to be detected,
wherein a partial sequence at the 5′ end of the third oligonucleotide strand of the third probe is complementarily paired with at least another partial sequence at the 3′ end of the second oligonucleotide strand of the second probe, to form a double-stranded region.

19. The method according to claim 13, wherein the ligation treatment is performed in the presence of a ligase,
optionally, the ligase is a Taq DNA ligase.

20. The method according to claim 16, wherein said capturing is performed in the presence of a streptavidin magnetic bead.
